SEQUENCE LISTING (36 sequences)

SEQ ID NO: 1 - RNA, 23 nt, Homo sapiens
agcuacauugucugcuggguuuc

SEQ ID NO: 2 - RNA, 24 nt, Homo sapiens
agcuacaucuggcuacugggucuc

SEQ ID NO: 3 - DNA, 1669 bp, Artificial Sequence
  Description of Artificial Sequence Synthetic polynucleotide
acttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaa

SEQ ID NO: 4 - RNA, 23 nt, Homo sapiens
ucgauguaacagacgacccaaag

SEQ ID NO: 5 - RNA, 24 nt, Homo sapiens
ucgauguagaccgaugacccagag

SEQ ID NO: 6 - DNA, 901 bp, Artificial Sequence
  Description of Artificial Sequence Synthetic polynucleotide
taagtgagaagacaaaatgaagaggaataaggttgtaaggcagtatgattaggcttgtgggtgctatgccttctgataataattatgaacagaaatagaagccaaaaagtaaacaacatgtaagttgtcaatctttgacagttgaggcagggagaaggaaggaaggatgacattacaccttatctctggtttactaggctggtgtgtgagaccatttgggtgaaatcgtattgaaatcattcattgctgaggtgatcagctttcttgcggtcctttctctgcactctattcaatgataaactccactggtttatacctcctggaaaacagttattcagaaacattataggggtagcattggtgagacagccaatggagaacatgtttccaggtagcctgaaacccagcagacaatgtagctgttgcctaacgaacacagaaatctacattgtatgccaggttcatgccccagacctggatgttcagcttgcaagtaattctcacatactatttcaacacaactgcctactgcattcaagatttcaaaattggcatttgtcttttctaccacaaggaaaagaaaaccaacagtcagaaatgctgggacttacctactaccaacaaaatttccttaaaccatcatatcatcaagttgtcatcatttaatcatgacatcatgttgaaaatgccatctaaaagtcaagagatgaaaaagctggatggaaggaaggtcggatagataaactggtaggtacgtgggcaggtgggtggatggatggatggatgggtggatggataaatgggtggatgggtaggggtagataggtaggtaggtagatagatgaatagattgatctatccaaatgatacctttcataggggggaaagagaaaaaaagagg

SEQ ID NO: 7 - DNA, 1092 bp, Artificial Sequence
  Description of Artificial Sequence Synthetic polynucleotide
tggaaggaaggtcggatagataaactggtaggtacgtgggcaggtgggtggatggatggatggatgggtggatggataaatgggtggatgggtaggggtagataggtaggtaggtagatagatgaatagattgatctatccaaatgatacctttcataggggggaaagagaaaaaaagaggtaaaacaaaaacaggtaagaggtaaaacatggataaatatccagattcctcccccttgtagtattgaaagcaaatagacctcatcattcataaaaccttgaaggttcccaagccccagctgataatgttggacttaacaccctagaacttgactctctcctctctctctgtctctctctctctctctctctctttgtgtgtgtgtgtgagtgtgtgtgtgtgtgtgtgtgtgtgtgtgtgtgtgtaattcaaggtaaagttttcattattaaagactgcccaataatctctctcaggacactgaagcagaagctagaagatgccatcagagacccagtagccagatgtagctgctgattacgaaagacaggatctacactggctactgagccattgagggtacctacaccttccagcagctgggtgatcctttgccttctggggaggggctctgtggaagaaaaaagaagatatcagatttcaattgcacattttctttggatcatgaattgacattttgacaatattcccatgtacgtaattttaaacaacctctggatttatttacttatccaacagatactgactgagtgacattaaataaagtgccacatattttcttaggtgctggagatccagcagcaaacaaaaaaagtggaaaatctctatacttctacagcatacatgattccttgtgacaaaatctacaatcaattatttggtatgttttgcagtaaaatggaaatatacacaagagaaaaacaaacaagcaaacaaaaaaccaatctaccattttattgtggcttggagcatttttgttgcttcctgtcactttaacttgggtaatctagcaatgatgcacatatgtaaagtaaattgcagttaaaaaattctttcctgttacgaactga

SEQ ID NO: 8 - RNA, 31 nt, Artificial Sequence
  Description of Artificial Sequence Synthetic oligonucleotide
uaaauuuuacccuuuagacuguagccuggau

SEQ ID NO: 9 - RNA, 31 nt, Artificial Sequence
  Description of Artificial Sequence Synthetic oligonucleotide
acagagacuuggcagccagaaauauccuccu

SEQ ID NO: 10 - RNA, 22 nt, Homo sapiens
cagugcaauguuaaaagggcau

SEQ ID NO: 11 - RNA, 22 nt, Homo sapiens
cagugcaaugaugaaagggcau

SEQ ID NO: 12 - RNA, 22 nt, Homo sapiens
gucacguuacaauuuucccgua

SEQ ID NO: 13 - RNA, 22 nt, Homo sapiens
gucacguuacuacuuucccgua

SEQ ID NO: 14 - DNA, 22 bp, Artificial Sequence
  Description of Artificial Sequence Synthetic probe
aaacccagcagacaatgtagct

SEQ ID NO: 15 - DNA, 23 bp, Artificial Sequence
  Description of Artificial Sequence Synthetic probe
agacccagtagccagatgtagct

SEQ ID NO: 16 - DNA, 36 bp, Artificial Sequence
  Description of Artificial Sequence Synthetic probe
tggtagcagaggatggtttcgatccatcgacctctg

SEQ ID NO: 17 - DNA, 24 bp, Artificial Sequence
  Description of Artificial Sequence Synthetic primer
gcagtatgattaggcttgtgggtg

SEQ ID NO: 18 - DNA, 24 bp, Artificial Sequence
  Description of Artificial Sequence Synthetic primer
catccacccatccatccatccatc

SEQ ID NO: 19 - DNA, 25 bp, Artificial Sequence
  Description of Artificial Sequence Synthetic primer
gtaggtagaatagatgaatagattg

SEQ ID NO: 20 - DNA, 24 bp, Artificial Sequence
  Description of Artificial Sequence Synthetic primer
catgtatgctgtagaagtatagag

SEQ ID NO: 21 - DNA, 25 bp, Artificial Sequence
  Description of Artificial Sequence Synthetic primer
ctcgagcgtcttagtccaaacccag

SEQ ID NO: 22 - DNA, 22 bp, Artificial Sequence
  Description of Artificial Sequence Synthetic primer
ctcgagcaaggacaaaagatct

SEQ ID NO: 23 - RNA, 23 nt, Homo sapiens
cuuugggucgucuguuacaucga

SEQ ID NO: 24 - RNA, 24 nt, Homo sapiens
cucugggucaucggucuacaucga

SEQ ID NO: 25 - RNA, 22 nt, Homo sapiens
uacgggaaaauuguaacgugac

SEQ ID NO: 26 - RNA, 22 nt, Homo sapiens
uacgggaaaguaguaacgugac

SEQ ID NO: 27 - RNA, 22 nt, Artificial Sequence
  Description of Artificial Sequence Synthetic oligonucleotide
augcccuuuuaacauugcacug

SEQ ID NO: 28 - RNA, 22 nt, Artificial Sequence
  Description of Artificial Sequence Synthetic oligonucleotide
augcccuuucaucauugcacug

SEQ ID NO: 29 - RNA, 23 nt, Artificial Sequence
  Description of Artificial Sequence Synthetic oligonucleotide
uuaguuuggauucuuauguagca

SEQ ID NO: 30 - RNA, 24 nt, Artificial Sequence
  Description of Artificial Sequence Synthetic oligonucleotide
cuuaguuuggauucuuauguagca

SEQ ID NO: 31 - RNA, 22 nt, Artificial Sequence
  Description of Artificial Sequence Synthetic oligonucleotide
uauucccaauagcaacguagcu

SEQ ID NO: 32 - RNA, 22 nt, Artificial Sequence
  Description of Artificial Sequence Synthetic oligonucleotide
uauucccaauagcaacguagcu

SEQ ID NO: 33 - RNA, 22 nt, Artificial Sequence
  Description of Artificial Sequence Synthetic oligonucleotide
uauuugcaguucaccugcacuu

SEQ ID NO: 34 - RNA, 22 nt, Artificial Sequence
  Description of Artificial Sequence Synthetic oligonucleotide
cuggaaguaaccauuugcacug

SEQ ID NO: 35 - RNA, 22 nt, Artificial Sequence
  Description of Artificial Sequence Synthetic oligonucleotide
uauuugcaguucaccugcacuu

SEQ ID NO: 36 - RNA, 22 nt, Artificial Sequence
  Description of Artificial Sequence Synthetic oligonucleotide
cuggaaguaaccauuugcacug

(19) United States
(12) Patent Application Publication
(10) Pub. No.: US 2009/0215862 A1
(43) Pub. Date: Aug. 27, 2009

(54) Micro rna

(75) Inventors: Cesare Peschle, Rome (IT); Nadia Felli, Rome (IT)

Correspondence Address: FOLEY & LARDNER LLP, P.O. BOX 80278, SAN DIEGO, CA, 92138-0278, US

(73) Assignee: ISTITUTO SUPERIORE DI SANITA

(21) Appl. No.: 11/918,459

(22) PCT Filed: Apr. 12, 2006
(86) PCT No.: PCT/EP2006/003899

§ 371 Date: Feb. 7, 2008

(30) Foreign Application Priority Data
Apr. 15, 2005 (GB) 0507679.9

(51) Int. Cl. A61K 31/7088; C12N 15/63; A61P 35/00

(52) U.S. Cl. 514/44 A; 435/320.1

(57) ABSTRACT

Micro RNA capable of interacting with the 3′ untranslated region of kit protein mRNA is useful in treating kit-dependent tumours, and inhibitors therefor are useful in treating suppressed haematopoiesis in cancer patients or abnormal erythropoiesis in β-thalassemia, for example.

The present invention relates to the use of micro RNAs in therapy.

Micro RNAs (miRs) are a recently discovered class of small (˜22nt) RNAs, which plays an important role in the negative regulation of gene expression by base-pairing to complementary sites on the target mRNAs (1). MiRs, first transcribed as long primary transcripts (pri-miRs), are processed in the nucleus by the RNase III enzyme Drosha to generate a 60-120 nucleotide precursor containing a stem-loop structure, known as pre-miR (2). This precursor, exported into the cytoplasm by the nuclear export factor Exportin-5 and the Ran-GTP cofactor, is finally cleaved by the RNase enzyme Dicer to release the mature miR (3).

MiRs mostly bind to the 3′ untranslated regions (UTR) of their target mRNAs. This process, requiring only partial homology, leads to translational repression. Target mRNAs which are more stringently paired may be cleaved (4, 5).

In excess of 300 miRs have so far been identified in eukaryotes. Generally, miRs are phylogenetically conserved (6-9). Their expression pattern is often developmentally determined and/or tissue-specific, although some miRs are steadily expressed throughout the whole organism (10). Growing evidence indicates that miRs are involved in basic biological processes, e.g.: cell proliferation and apoptosis (11,12); neural development and haematopoiesis (13); fat metabolism; stress response; and cancer (14-16), via the targeting of key functional mRNAs. Little is known of the functional role of miRs in mammals, and even less on the targets in mammals (13,16).

We have now found that treatment of CD34+ cells with two naturally occurring micro RNAs, miR221 and miR222, causes impaired proliferation and accelerated differentiation of erythroid cells, coupled with down-modulation of kit protein, while levels of kit mRNA are unaffected, and that miR221 and miR222 gene transfer blocks proliferation of the TF1 erythroleukemic cell line. Treatment with anti-miR221 and miR222 oligonucleotides causes the opposite effect.

Similar results have also been found with miR130a and miR130b.

Thus, surprisingly, we have now found that miR 221, miR 222, miR130a and miR130b can each inhibit or block translation of kit mRNA.

Thus, in a first aspect, the present invention provides the use of antisense RNA specific for all or part of the 3′ untranslated region of kit protein mRNA in therapy.

The 3′ untranslated region (UTR) of human kit protein mRNA is provided as accompanying SEQ ID NO. 3. Antisense RNA may be specific for any part of the 3′ UTR of kit protein mRNA, and it will be appreciated that the 3′ UTR may vary slightly from individual to individual.

In addition, as noted above, miR need not be 100% faithful to the target, sense sequence. Indeed, where they are 100% faithful, this can lead to cleavage of the target mRNA through the formation of dsRNA. While the formation of dsRNA and cleavage of kit protein mRNA is included within the scope of the present invention, it is not a requirement that the antisense RNA be 100% faithful to the target sequence, provided that the antisense RNA is capable of binding the target 3′ UTR to inhibit or prevent translation.

Thus, it will be appreciated that the antisense RNA of the present invention need only exhibit as little as 60% or less homology with the target region of the 3′ UTR. More preferably, the antisense RNA exhibits greater homology than 60%, such as between 70 and 95%, and more preferably between 80 and 95%, such as around 90% homology. Homology of up to and including 100%, such as between 95 and 100%, is also provided.

The antisense RNA of the present invention may be as long as the 3′ UTR, or even longer. However, it is generally preferred that the antisense RNA is no longer than 50 bases, and it may be a short as 10 bases, for example. More preferably, the antisense RNA of the present invention is between about 12 bases and 45 bases in length, and is more preferably between about 15 and 35 bases in length.

Preferred miRs are miR 221 and miR 222. Their mature sequences are shown hereinafter as SEQ. ID NO's 1 and 2, and have a mature length of 23 or 24 bases. Thus, a particularly preferred length is between 20 and 25 bases, and especially 23 or 24.

The area of the 3′ UTR to be targeted may be any that prevents or inhibits translation of the ORF, when associated with an antisense RNA of the invention. The particularly preferred regions are those targeted by miR 221 and miR 222, and targeting either of these regions with antisense RNA substantially reduces translation of kit protein.

Regions of the 3′ UTR that it is preferred to target include the central region of the 3′ UTR and regions between the central region and the ORF. Such regions which are proximal to the ORF are particularly preferred.

Other kit mRNA sequences, such as the coding region for instance, may also be targeted.

It is preferred that the antisense RNA of the present invention is a short interfering RNA or a micro RNA.

As noted above, preferred miRs are miR 221 and miR 222. However, also preferred are miR130a and miR130b (SEQ ID NO's. 10 and 11), which have also been shown to work in a similar manner. It will be understood that reference to miR221 and miR222 made herein, therefore also includes reference to miR130a and miR130b, unless otherwise apparent.

The present invention further provides mutants and variants of these miRs. In this respect, a mutant may comprise at least one of a deletion, insertion, inversion or substitution, always provided that the resulting miR is capable of interacting with the 3′ UTR to inhibit or prevent translation of the associated coding sequence. Enhanced homology with the 3′ UTR is preferred. A variant will generally be a naturally occurring mutant, and will normally comprise one or more substitutions.

Particularly preferred stretches of the microRNA of the present invention correspond to the so-called “seed” sequences highlighted in FIG. 8, in particular 5′-GCTACAT-3′ of miR 221 and 222 (ntd positions 2-8 in SEQ ID NOs. 1 and 2) according to algorithm Targetscan I, which matches exactly, i.e. corresponds or hybridises under highly stringent conditions to, ntds 3982-3988 in the kit 3′ UTR (SEQ ID NO. 3) and is associated with additional flanking matches (again, see FIG. 8) The seed sequence is conserved in mouse and rat.

Also preferred are the corresponding sequences in miR-130a and miR-130b.

It will be appreciated that reference to any sequence encompasses mutants and variants thereof, caused by substitutions, insertions or deletions, having levels of sequence homology (preferably at least 60%, more preferably at least 70%, more preferably at least 80%, more preferably at least 90%, more preferably at least 95%, more preferably at least 99%, and most preferably at least 99.5% sequence homology), or corresponding sequences capable to hybridising to the reference sequence under highly stringent conditions (preferably 6×SSC).

As can bee seen in FIG. 6, miR 222 and 130a physically interact with Kit 3′UTR. This shows that treatment with anti-miR 221 and 222 sequences markedly upmodulates kit protein. The same action has been shown by infecting the cells with “decoy” sequences in lentiviral vector (these sequences include the “seed” sequence matching 221/222, as well as the closeby “ancillary” matches). Noteworthily, these antisense “decoy” sequences match for <50% miR 221/222. The effect of anti-miR 221 and 222 sequences on kit protein level has been validated in functional assays (similar or identical to those presented in the Examples for miR 221 and 222).

FIGS. 6 and 8 show that miR 130a (see FIG. 6) and miR 130b (almost identical to miR 130b except for 2 nucleotides, see FIG. 8; see also FIG. 6 legend) also directly interact with the kit 3′ UTR in the same way that miR 221 and 222 do.

The antisense RNAs of the present invention may be provided in any suitable form to the target site. In this respect, the target site may be in vivo, ex vivo, or in vitro, for example, and the only requirement of the antisense RNA is that it interacts with the target 3′ UTR sufficiently to be able to inhibit or prevent translation of the kit ORF.

The antisense RNA may be provided directly, or a target cell may be transformed with a vector encoding the antisense RNA directly, or a precursor therefor. Suitable precursors will be those that are processed to provide a mature miR, although it is not necessary that such precursors be transcribed as long primary transcripts, for example.

Where the antisense RNA is provided directly, then this may be provided in a stabilised form such as is available from Dharmacon (www.dharmacon.com, Boulder, Colo., USA).

A large number of microRNAs are known from WO 2005/013901, the patent specification of which alone is over 400 pages. This publication discloses, in particular, the sequences of miR221, miR222, miR130a and miR30b. However, no specific function is provided therefor. Similarly, WO 2005/017145 also discloses at least one of the above mentioned miRNAs and provides it with a role in gene expression.

U.S. Pat. No. 5,989,849 and U.S. Pat. No. 5,734,039 disclose antisense RNA that target the kit mRNA transcript. However, this is not by means of naturally-occurring sequences, but rather synthetic nucleotides, which is less desirable. A similar position is described in WO 92/19252.

Indeed, use of RNA interference (RNAi) to disrupt kit gene expression is well known, see for instance Demir et al (Blood, Vol. 96, 2000) and Yamanishi et al (Jpn. J. Cancer Res., Vol. 87, 1996, bp. 534-542).

Thus, although microRNAs are known, as is targeting kit protein expression by antisense RNA technology, such as interference RNA, we are the first to establish that naturally-occurring RNA sequences, in particular miR 221, 222, 130a and 130b, or inhibitors thereof, are in fact capable of modulating the expression of kit protein.

Insofar as miR 221 and miR 222 miR130a and miR130b are known, and any stabilised versions thereof, such as provided by Dharmacon are known, then the present invention does not extend to these compounds per se. However, the present invention extends to these and all other antisense RNAs provided by the present invention, for use in therapy and other processes.

More particularly, the present invention provides the use of antisense RNA specific for all or part of the 3′ untranslated region of kit protein mRNA in therapy.

The nature of the therapy is any that is affected by expression of kit protein. In particular, antisense RNAs of the present invention may be used in the treatment of GIST (gastro-intestinal stromal tumour), kit-dependent acute leukaemias and other kit-dependent tumours.

Solid, non-diffuse tumours may be targeted by direct injection of the tumour with a transforming vector, such as lentivirus, or adenovirus. If desired, the virus or vector may be labelled, such as with FITC (fluorescein isothiocyanate), in order to be able to monitor success of transformation.

In addition to the examples provided in the present application, the invention has been proven not only to inhibit normal erythropoiesis and erythroleukemic cell growth via kit receptor down-modulation, but also to have a role in papillary thyroid carcinoma (PTC), see for instance Felli et al (PNAS, 13^{th} Dec. 2005, Vol. 102, No. 50, P. 18081-18086) and He et al (PNAS, 27^{th} Dec. 2005, Vol. 102, No. 52, Pages 19075 to 19080).

Thus, it is also preferred that the present invention is used in the modulation of erythropoiesis and/or the prophylaxis or treatment of erythroleukemic cell growth, cancer in general, especially papillary thyroid carcinoma, preferably by via kit receptor down-modulation.

For the treatment of a more diffuse condition, then systemic administration may be appropriate, and antisense RNA may be administered by injection in a suitable vehicle, for example.

Levels of antisense RNA to be administered will be readily determined by the skilled physician, but may vary from about 1 μg/kg up to several hundred micrograms per kilogram.

The present invention further provides miR 221 and miR 222 inhibitors, and their use in therapy. These are referred to as “sense inhibitors” in that they are complementary, at least in part, to the antisense miRNA of the present invention.

miR 221 and miR 222 are naturally occurring, and high levels of these micro RNAs inhibit erythropoiesis, and this effect can be undesirable, such as with cancer patients undergoing chemotherapy, which can repress erythropoiesis.

Accordingly, the present invention provides the use of an miR 221 and miR 222 inhibitor in therapy.

Also provided is the use of a sense or antisense polynucleotide according to present invention in the manufacture of a medicament for the treatment or prophylaxis of the conditions specified herein.

Preferably, where one such inhibitor is used, a second inhibitor to the other miR is also provided, in order to enhance kit protein expression. Thus, it is preferred to provide an inhibitor both for miR 221 and for miR 222 in any such therapy.

Suitable inhibitors for miR 221 and miR 222 include antibodies and sense RNA sequences capable of interacting with these miRs. Such sense RNAs may correspond directly to the concomitant portion of the 3′ UTR of kit mRNA, but there is no requirement that they do so. Indeed, as miRs frequently do not correspond entirely to the 3′ UTR that they target, while the existence of dsRNA often leads to destruction of the target RNA, then it is a preferred embodiment that the inhibitor of miR 221 or of miR 222 is entirely homologous for the corresponding length of miR 221 or miR 222. The length of the inhibitor need not be as long as miR 221 or miR 222, provided that it interacts sufficiently at least to prevent either of these miRs interacting with the 3′ UTR or kit mRNA, when so bound.

The same results have been obtained with anti-miR 130a (SEQ ID NO. 27) or 130b sequences (SEQ ID NO. 28) or mutants, variants thereof or sequences comprising any of these. Thus, where reference is made to miR 221 and miR 222 inhibitors, it will be appreciated that this also includes reference to miR 130a and miR 130b inhibitors, which are also preferred and are preferably the sequences described above.

Conditions treatable by miR 221 and miR 222 inhibitors include suppressed haematopoiesis in cancer patients and β-thalassemia and other β-haemoglobin diseases.

In β-thalassemia and other β-haemoglobin diseases, for instance sickle cell anemia, miR 221 and miR 222 inhibitors may be used to enhance the level of γ-globin synthesis, thus leading to a therapeutic effect.

Such inhibitors may also be used for the potentiation of ex vivo expansion of haematopoietic stem/progenitor cells and for the enhancement of the proliferative and anti-apoptotic effects of kit in non-haematopoietic cells, whether such cells be of a normal or abnormal phenotype.

Preferred methods of delivery of the antisense miRNA or sense inhibitors may be by any gene therapy method known in the art, as will be readily apparent to the skilled person. Such methods include the so-called “gene-gun” method or delivery within viral capsids, particularly adenoviral or lentiviral capsids encapsulating or enclosing said polynucleotides, preferably under the control of a suitable promoter.

Preferred means of administration by injection include intravenous, intramuscular, for instance. However, it will also be appreciated that the polynucleotides of the present invention can be administered by other methods such as transdermally or per orally, provided that they are suitably formulated.

We have now established that treatment of CD34+ cells with miR221 and miR222, via oligomer transfection or lentiviral vector infection (SEQ ID NO's. 4, 5, 6 and 7), causes impaired proliferation and accelerated differentiation of erythroid cells, coupled with down-modulation of kit protein. Levels of kit protein mRNA are unaffected. In addition, transplantation experiments in NOD-SCID mice reveal that miR221 or miR222 treatment of CD34+ cells impairs their engraftment capacity. Further, miR221 and miR222 gene transfer blocks proliferation of the TF1 erythroleukemic cell line, a line that expresses the kit receptor.

Thus, in human erythropoiesis, reduction in levels of miR221 and miR222 microRNA serves to unblock kit protein production at the translational level, thereby playing a pivotal role in the expansion of differentiating erythroid cells. An inhibitory role in early haematopoiesis is also likely. Furthermore, over-expression of miR221 and miR222 inhibits proliferation of erythroleukemic cells expressing the kit receptor.

Treatment with anti-miR221 and miR222 oligonucleotides or sequences (SEQ ID NO's. 8, 9 and 3), i.e., antisense miR sequences, “decoy” miR target sequences, results in the opposite effect, compared with treatment with miR221 and miR222.

These results indicate the possibility of modulating the level of kit protein at biological and therapeutic levels by means of miR or anti-miR221 and anti-miR222 treatment, for example. This is of importance, as kit is the receptor of stem cell factor (SCF), considered the key growth factor in the proliferation of primitive haematopoietic and erythropoietic cells. Furthermore, constitutive activation of kit has an oncogenic effect in diverse neoplasias, e.g., some acute leukaemias and GIST (gastro-intestinal stromal tumour).

Our results do not preclude the possibility that miR221 and miR222 hamper early haematopoiesis and erythropoiesis by blocking the translation of other key functional proteins, i.e., bioinformatics analysis suggests that GATA-2 transcription factor, Bcl2 anti-apoptotic factor, member(s) of the E2F cell cycle proteins may be targeted by miR221 and miR222.

It will be appreciated that the kit receptor plays a key functional role in non-haematopoietic tissues, such as in smooth muscle progenitors, neural progenitors, melanocytes, etc. Therefore, the functional effect of miR221 and miR222 to inhibit kit mRNA translation is not restricted to early haematopoiesis and erythropoiesis, and its use in respect of other tissues is also contemplated.

Thus, miR221 and miR222 play a key functional role in early haematopoiesis and erythropoietic differentiation/maturation, at least in part via unblocking of kit receptor mRNA translation. The results further suggest that miR221 and miR222 may modulate the growth of kit+ leukaemic cells. The functional role of miR221 and miR222 may be extended to other kit+ non-haematopoietic tissues of either normal or abnormal type, e.g., smooth muscle cell progenitors (Cajal cells) and GIST tumours.

Therefore, one of the advantages of the present invention is that naturally-occurring microRNA sequences, which are antisense to the 3′ UTR of the kit mRNA, or sense sequences which inhibit said antisense microRNAs, can be used to modulate the level of kit protein expression.

Also provided is a “test kit” capable of testing the level of expression of the kit protein such that the physician or patient can determine whether or not levels of the kit protein should be increased or decreased by the sense or antisense sequences of the present invention.

The present invention also encompasses a polynucleotide sequence, particularly a DNA sequence, which encodes the microRNAs of the present invention, operably linked to a suitable first promoter so that the MicroRNAs can be transcribed in vivo. Similarly, the present invention also provides a polynucleotide, particularly DNA, providing polynucleotides encoding the sense microRNA inhibitors of the present invention, also operably linked to a suitable second promoter for in vivo expression of said sense microRNA inhibitors.

In particular, it is also preferred that the first and second promoters mentioned above can be controlled by a third element, such that the level of expression of the antisense microRNA and the level of expression of the sense microRNA inhibitors can be controlled in a coordinated manner. In this regard, it is preferred that a feedback mechanism is also included for establishing this level of control.

Chimeric molecules are also provided, consisting of a polynucleotide according to the present invention, i.e. the antisense MicroRNAs or the sense microRNA inhibitors, linked to a second element. The second element may be a further polynucleotide sequence or may be a protein sequence, such as part or all of an antibody. Alternatively, the second element may have the function or a marker so that the location of microRNAs can be followed.

The present invention will now be further illustrated by the following, non-limiting Examples.

### EXAMPLES

### Materials and Methods

### Cell Culture

(a) Unilineage Erythropoietic Culture of CD34+ Cells from Cord Blood

Cord blood (CB) was obtained from healthy, full-term placentas according to institutional guidelines. Low-density mononuclear cells (MNCs) (less than 1.077 g/mL) were isolated by Ficoll-Hypaque density-gradient centrifugation, and CD34+ cells were purified by MACS column (Miltenyi, Bergish Gladbach, Germany).

Purified HPC were grown in foetal calf serum (FCS)-free medium (10⁵ cells/ml) in a fully humidified 5% CO₂, 5% O₂, 90% N₂ atmosphere and were induced to unilineage erythropoietic differentiation by an erythroid-specific HGF cocktail [saturating dosage of Epo (3 U/ml), low-dose of IL3 (0.01 U/ml) and GM-CSF (0.001 ng)]. The HGF cocktail was supplemented or not with KL (100 ng/ml).

To evaluate erythropoietic cell proliferation, CD34+ progenitor cells were grown in triplicate in 24-well plates in 0.5 mL of serum-free medium containing the erythroid-specific HGF cocktail supplemented or not with 100 ng/mL KL. Cells were counted every 2-3 days and diluted at 2×10⁵ cells/mL. For morphology analysis, cells were harvested from day 8 to day 29, smeared on glass slides by cytospin centrifugation and stained with standard May-Grunwald-Giemsa.

(b) TF1 and HL60 Cell Culture

Human erythroleukemia-derived cell line TF1 was obtained from the American Type Culture Collection. Cells were routinely grown in RPMI 1640 medium (Gibco), supplemented with 10% FCS (Gibco) and 2 ng/ml GM-CSF (Peprotech).

Promyelocytic cell line HL-60 was maintained in RPMI 1640 medium (Gibco) supplemented with 10% FCS (Gibco). Cells were grown at 37° C. in a humidified 5% CO₂ incubator.

miR221 and miR222 Expression

(a) Microarray and Bioinformatic Analysis

Microarray analysis was performed as described (17). Briefly, labelled targets from 5 μg of total RNA were used for hybridisation on KCC/TJU microarray chip containing 368 probes in triplicate, corresponding to 161 human and 84 mouse precursors miRNA genes. The probes (40-mer oligonucleotides) are spotted by contacting technologies and covalently attached to a polymeric matrix. The microarray were hybridised in 6×SSPE/30% formamide at 25° C. for 18 h, washed in 0.75×TNT (Tris-HCl/sodium chloride/Tween) at 37° C. for 40 min, and processed by using direct detection of the biotin-containing transcripts by Streptavidin—Alexa647 conjugate. Processed slides were scanned by using a Perkin Elmer ScanArray XL5K Scanner. The expression level were analysed by QUANTARRAY software (Perkin Elmer).

Raw data were normalised and analysed using the GENESPRING software version 6.1.1 (Silicon Genetics, Redwood City, Calif.). The average value of three spot replicates of each miRNA was transformed (to convert any negative value to 0.01) and normalised using a per-chip 50^{th} percentile method that normalises each chip on its median, allowing comparison among chips.

(b) Northern Blot

Total RNA isolation was performed using the Acid Phenol-Guanidinium Thiocyanate-Chloroform protocol (18). RNA samples (25 μg each) were run on 15% acrylamide denaturing Criterion precast gels (Bio-Rad) and then transferred onto Hybond-n+membrane (Amersham Pharmacia Biotech). The hybridisation was performed with specific probes, previously labelled with [γ]-³²PATP, at 37° C. in 0.1% SDS/6×SSC overnight. Membranes were washed at room temperature twice with 0.1% SDS/2×SSC. Human tRNA for initiator methionine (Met-tRNA) was used as loading control.

The probes used are:

(SEQ ID NO. 14)

miR221-5′-AAACCCAGCAGACAATGTAGCT-3′

(SEQ ID NO. 15)

miR222-5′-AGACCCAGTAGCCAGATGTAGCT-3′

(SEQ ID NO. 16)

Met-tRNA-5′TGGTAGCAGAGGATGGTTTCGATCCATCGACCTCTG-
3′.

Blots were stripped at 65° C. in 0.1% SDS/0.1×SSC for 15 min and reprobed.

The expression levels were analysed by the Scion Image Software (Scion Corporation USA, www.scioncoro.com).

C-Kit Expression

(a) Real Time PCR

Total RNA was extracted by the standard guanidinium thiocyanate-CsCl method in the presence of 12 μg of Escherichia coli rRNA or by Rneasy kit (Quiagen) and reverse-transcribed with oligo (dT) as a primer. RT-PCR was performed by TaqMan technology, using the ABI PRISM 7700 DNA Sequence Detection System (Applied Biosystems, Foster City, Calif., USA) according to standard procedures (19). Thermal cycling was performed using 40 cycles of 95° C. for 15 s and 60° C. for 1 min. Glyceraldeyde-3-phosphate dehydrogenase (GAPDH) and 18S RNA were selected as endogenous controls to correct for potential variation in RNA loading or efficiencies of the reverse transcription or amplification reaction. Original input RNA amounts were calculated with relative standard curves for both the RNA of interest and the endogenous controls. Duplicate assays were performed with RNA samples obtained from at least two independent experiments. Commercial ready-to-use primers/probe mixes were used (Assays on Demand Products, Applied Biosystems, Foster City, Calif., USA).

(b) Western Blot and FACS Analysis

Total c-kit protein expression was analysed by Western blotting. Briefly, cells were washed with PBS and lysed with lysis buffer (20 mM Tris, pH 7.2, 150 mM NaCl, 1% NP-40, protease inhibitor cocktail). Debris were pelleted by centrifugation and supernatants were resolved by SDS-PAGE and Western blotting using an anti-kit antibody (R&D) and a secondary anti-goat IgG antibody peroxidase conjugate (Chemicon). The expression levels were analysed by the Scion Image Software (Scion Corporation USA, www.scioncoro.com).

Membrane-bound c-kit protein expression was analysed by fluorescence-activated cell sorting (FACS), 24, 48 and 72 hours after transfection. 1×10⁵ cells were washed with PBS, pre-incubated with 40 μg/mL of mouse IgG (Sigma) and then incubated with CyChrome conjugated anti-c-kit or anti-IgG control antibodies (BD Pharmingen). After washing cells were analysed by FACS.

Luciferase Assay

Twenty-four hours after plating HeLa cells to a density of 2×10⁵ cells/well in 24-wells plates, they were co-transfected with 0.1 μg of pGL3-3′-UTR plasmid and 0.3 μg of either Tween, Tween-miR221, Tween-miR222 or Tween-miR221+Tween-miR222 with Lipofectamine 2000 (Invitrogen) following the manufacturer's instructions.

After transfecting the cells for 48 h, the cells were washed and lysed with the Passive Lysis Buffer (Promega), and their luciferase activity was measured using the Femtomaster FB 12 (Zylux Corp.) as indicated by the manufacturer's protocol.

The relative reporter activity was obtained by normalising it to the pGL3-3′-UTR/Tween cotransfection.

All experiment were carried out in 6 independent experiment, and results are presented as mean+/−SD.

Cell Transfection with miR221 and miR222 Oligomers

(a) Oligomers

Stability Enhanced siRNA miR221, miR222 and the non-targeting negative control, that has at least 4 mismatches with all known human and mouse genes (referred as miR221, miR222 and miRCont, respectively), or FITC-conjugated siRNAs, were purchased from Dharmacon and prepared according to the manufacturer's instructions.

(b) Transfection Procedure

On the day of transfection, TF1 cells were seeded at 2×10⁵ cells/ml in 24-well plates in antibiotic-free media and transfected with miRNAs at a concentration of 40 nM or 80 nM. Cord blood CD34+ progenitor cells were cultured in erythroid medium plus KL (100 ng/ml) and transfected on day 4 of erythroid differentiation. On the day of transfection cells were seeded at 1.2×10⁵ cells/ml in 24-well plates in antibiotic-free media and transfected with miRNAs at a concentration of 160 nM. Transfections were done with Lipofectamine 2000 according to the manufacturer's instructions (Invitrogen). Percentage of FITC-positive cells was evaluated 16 hours after transfection with FACSCalibre flow cytometer and CellQuest software (Becton Dickinson, Oxford, United Kingdom).

Cell Infection with Lentiviral Vectors

(a) Plasmids and Constructs

Tween-miR

MiR221 and miR222 precursors cDNA were first PCR-amplified from a human BAC clone using Accuprime Taq DNA polymerase High Fidelity (Invitrogen).

The primers used for the amplification of miR221 were:

Forward

(SEQ ID NO. 17)

5′-GCAGTATGATTAGGCTTGTGGGTG-3′
Reverse

(SEQ ID NO. 18)

5′-CATCCACCCATCCATCCATCCATC-3.′
(NCBI reference: NT_011568 from 2014600 to
2015500)

The primers used for the amplification of the miR222 were:

Forward

(SEQ ID NO. 19)

5′-GTAGGTAGAATAGATGAATAGATTG-3′
Reverse

(SEQ ID NO. 20)

5′-CATGTATGCTGTAGAAGTATAGAG-3′.
(NCBI reference: NT_011568 from 2015320 to
2016411)

Both of the cDNA's length was approximately 1063 bp.

MiR221 and miR222 were then cloned in the pCR 2.1-TOPO vector (Invitrogen) using the manufacturer's instructions.

TOPO-miR's vectors were then digested with BamHI enzyme (NEB) and filled with T4 DNA Polymerase (NEB). The fragment obtained from the digestion of TOPO-miR vector with XhoI (NEB), was then inserted, in frame, into the self inactivating transfer vector plasmid, pRRL-CMV-PGK-GFP-WPRE (20) called Tween, previously digested with XbaI (NEB), filled with T4 DNA Polymerase (NEB), digested again with XhoI (NEB), and treated with Calf Alkaline Phosphatase (CIP, NEB) for 30′ at 37 C.

PGL3-3′-UTR

The 3′-UTR from the c-Kit gene was cloned from human spleen genomic DNA (BioChain) using the forward primer 5′-CTCGAGCGTCTTAGTCCAAACCCAG-3′ (SEQ ID NO. 21), and the reverse 5′-CTCGAGCAAGGACAAAAGATCT-3′ (SEQ ID NO. 22), containing the XhoI endonuclease recognition site. The fragment obtained was cloned in the pCR 2.1-TOPO vector (Invitrogen), digested with XhoI (NEB), and subcloned in the pGL3-Promoter vector (Promega), previously digested with XhoI (NEB), and treated as described with Calf Alkaline Phosphatase, downstream the luciferase gene.

All the sequences were confirmed by automated sequencing performed by the Nucleic Acid Facility of the Kimmel Cancer Institute.

(b) Infection Procedure

Gene transfer was performed by using the lentiviral vector, Tween, variant of third-generation lentiviral vectors (20) (21), to simultaneously transduce both reporter and miRNA.

Lentiviral supernatants were produced by calcium phosphate transient cotransfection of a three-plasmid expression system in the packaging human embryonic kidney cell line 293T. The calcium-phosphate DNA precipitate was removed after 14-16 h by replacing the medium. Viral supernatant was collected 48 h filtered through 0.45 μm pore nitrocellulose filters, and frozen in liquid nitrogen (20). During infection CD34+ cells were plated at 5×10⁴ cells/ml, in a six-well plate in presence of viral supernatant. 4 μg/ml of polybrene was added to the viral supernatant to improve the infection efficiency. Cells were centrifuged for 45 min at 1,800 revolutions/min and incubated for 75 min in a 5% CO₂ incubator. After the infection cycles, CD34+ cells were washed twice and replated in fresh medium. Infection efficiency was evaluated after 48 h by flow cytometry.

NOD SCID Experiments

Breeding pairs of NOD/Ltsz scid/scid mice (NOD/SCID, originally obtained from Dr. Dominique Bonnet, Corriel Institute, Camden, N.J.) were housed in microisolator under pathogen-free conditions and received autoclaved food and acidified water at libitum. Seven- to 9-week-old mice received a sublethal dose of whole-body irradiation (350 cGy). Within 24 hours after irradiation, CB CD34+ cells transfected with miR 221 or 222 oligomers (see above) were injected in the tail vein in a volume of 200 μl, together with γ-irradiated (2000 cGy) CB CD34− accessory cells (1×10⁶ cells/mouse). Mice were sacrificed 6 weeks after cell transplantation and bone marrow (BM) cells were harvested from femurs and tibiae as described (22). Cells were stained with mouse anti-human CD45-FITC and CD34-PE MoAbs (R&D) and analysed on a FACSCalibur (B-D), excluding dead cells stained by 7-AAD (Sigma). Positive cells were identified by comparison with isotypic controls. The xenoengraftment level was expressed as percentage of human CD45+ cells/total nucleated cells. For multilineage engraftment analysis, cells were stained for lineage-specific human haematopoietic antigens. FITC-conjugates MoAbs included anti-human CD45 (R&D), CD15, CD19, CD3, CD16 (BD), PE-conjugated antibodies included: anti-human CD34 (R&D), Glicophorin-A, CD41, CD33, CD14, CD20, CD4, CD56 (B-D).

### Results

Microarray Analysis Reveals miR221 and miR222 Down-Modulation During Erythroid Differentiation in Unilineage CD34+ Cell Culture: Inverse Correlation of miR221/222 Expression and Kit Protein Level During Erythroid Differentiation (FIG. 1A, B)

Recent Studies Suggested that miRNAs Modulate Haematopoietic Lineage differentiation (13). In order to investigate the involvement of miRNAs in erythroid differentiation, we analysed their expression at discrete stages of unilineage erythroid culture of CB CD34+(FIG. 1A). The analysis was performed using a microarray chip containing as probes gene-specific 40 mer oligonucleotides, generated from 161 human and 84 mouse precursors miRNA (17). The expression profile revealed that miR221 and miR222 had statistically significant differences in expression levels during erythropoiesis. In fact, as shown in FIG. (1B), miR221 and miR222 are strongly expressed in CD34+ progenitor cells and they gradually decrease during erythroid differentiation. Northern blot analysis confirmed the data obtained using the microarray profiling system (FIG. 1B).

As miRs have been reported to play an important role in the negative regulation of gene expression, we hypothesised that miR221 and miR222 decline may promote erythropoiesis unblocking expression of key functional proteins. We searched for complementary sites in 3′UTRs of genes known to play a pivotal role in erythropoiesis, and, using multiple computational approaches, we noticed that c-kit protein could had been a putative target for miR221 and miR222. This observation prompted us to investigate the expression pattern of c-kit in unilineage erythroid culture. As expected, Western blot analysis showed that c-kit protein gradually increases during erythroid differentiation, reaching the highest level in late erythroblasts. Real time PCR clearly showed that c-kit mRNA is almost constantly expressed in the whole differentiation process (FIG. 1B). Accordingly to a post-transcriptional regulatory mechanism, the expression level of both miR221 and miR222 inversely correlate with the accumulation pattern of the c-kit target protein (FIG. 1B).

Kit Ligand (KL) Promotes c-Kit Protein Expression Via not Only Translational but Also Transcriptional Mechanisms (FIG. 1c)

KL (also termed stem cell factor, SCF) has been reported to promote the survival of haematopoietic progenitors and to delay erythroid differentiation. Western blot analysis revealed that c-kit receptor was markedly up-regulated in erythropoietic culture treated with KL (FIG. 1C), more than observed in cultures not supplemented with KL (FIG. 1B, C). Both miR221 and miR222 were down-modulated during erythroid differentiation in KL-treated culture (FIG. 1C), as observed in erythroid culture not supplemented with KL (FIG. 1B). However, real time PCR experiments revealed that c-kit mRNA was significantly increased upon KL treatment (not shown), thus indicating that in these culture conditions kit protein expression is also up-regulated at transcriptional level.

miR221 and miR222 Physically Interact with c-Kit 3′UTR (FIG. 2a)

In order to demonstrate the direct physical interaction between the 3′UTR of the c-kit mRNA, we inserted downstream the luciferase ORF the 1800 bp's 3′UTR portion of the c-kit messenger RNA. After the co-transfection of this reporter vector with either the Tween vector alone, a Tween vector encoding either miR221 or miR222, or both, we measured the relative reporter activity, normalising to the co-transfection with the empty Tween.

The relative luciferase activity observed was significantly diminished, compared to the empty vector, in both the miR221 and miR222 co-transfection. Interestingly, in the triple transfection with both the miR-encoding vectors, the reporter activity measured was even lower, indicating a cooperative effect of the two miRs, probably due to their different target sites on the c-kit 3′UTR.

Together these results indicate that the two miRs interfere with the c-kit mRNA translation via direct interaction with the 3′ untranslated region of the messenger.

miR221 and miR222 Oligomers Down-Modulate Kit Expression in TF1 Erythroleukemic Line (FIG. 2B)

In order to demonstrate that miR221 and miR222 modulate the expression of the c-kit protein, we analysed the expression of c-kit in cells transfected with miR221 and miR222. As a model system we chose the erythroleukemia-derived cell line TF1 which express high levels of c-kit (23) and, as expected, low levels of miR221 and miR222 by Northern blotting (data not shown). We transfected TF1 cells with double-stranded (ds) RNAs having the same sequence of respectively the mature miR221 and miR222 or with the non-targeting negative control (miRCont). Consistent with the prediction that c-kit expression is negatively regulated by miR221 and miR222, FACS analysis with a c-kit specific antibody revealed that the protein is strongly reduced in TF1 cells transfected with exogenous miR221, miR222 or both, relative to the same cells transfected with the negative control miR (FIG. 2B, panel a). The inhibition of c-kit expression was dose-dependent (FIG. 2B, panel c) and time-dependent, showing a maximum inhibition at 48 hours post-transfection (FIG. 2B, panel d). In order to monitor the uptake of the dsRNA by TF1 cells, we transfected cells with a FITC-conjugated miRNA and analysed by FACS 16 hours after transfection (FIG. 2B, panel b). As shown, a high percentage of cells were FITC-positive, indicating that dsRNA is efficiently transfected in these cells; moreover, apoptosis analysis showed that miRNA was not toxic for cells (data not shown). There was no difference in the transfection efficiency within different FITC-conjugated miRNAs (data not shown).

Down-modulation of c-kit expression was also demonstrated by Western blotting in TF1 cells transfected with miR221, miR222 or both at a concentration of 80 nM (FIG. 2B, panel e). The protein expression levels of actin were unaffected by the transfected miR221, miR222, miR221 plus miR222 and negative control miRNAs, indicating that miR221 and miR222 regulation is specific to c-kit. Since miRNAs are known to inhibit protein expression at translational level, we analysed c-kit mRNA expression in TF1 cells transfected with miRNAs at a concentration of 80 nM. As shown in FIG. 2B (panel f), c-kit mRNA expression levels, analysed by quantitative real-time PCR, were almost constant in the miR over-expressing cells compared to the Lipofectamine-alone treated cells or cells transfected with control miR. Although cells treated with miR222 or with miR221 plus miR222 showed a small decrease of c-kit mRNA expression, the entirety of the down-modulation was much less pronounced compared to the one observed at the protein level, indicating that the regulation of c-kit by miRNAs occurs at translational level.

Lentiviral Gene Transfer of miR221 and miR222 in TF1 Erythroleukemic Line Acts as Growth Repressor and c-Kit Repressor (FIG. 3a)

We investigated the effects of miR221 and miR222 gene transfer in TF-1 erythroleukemic cell line, expressing the c-kit receptor. We transduced TF-1 cells with a lentiviral vector encoding alternatively the miR221 and miR222 under the control of a CMV promoter, and a GFP reporter gene under the PGK promoter, to constantly monitor the number of infected cells. Empty vector was transduced as a negative control.

Sorted cells were cultivated in standard medium and cell proliferation measured at different times.

Evaluation of viable cell number revealed that TF-1 cells expressing miR221 or miR222 showed a reduced proliferative rate when compared with empty vector-transduced cells. We then analysed whether such enforced expression could interfere with c-kit protein expression, and a clear reduction was observed. Interestingly, as expected in case of a post-transcriptional mechanism, no relevant modulation of c-kit mRNA was observed by real time PCR.

Moreover, we investigated the effects of miR221 and miR222 gene transduction in HL-60 cell line, lacking the c-kit protein. As expected, no relevant difference was observed in their proliferative rate, compared to empty vector-transduced cells.

miR221 and miR222 Oligomer Treatment of CD34+ Cells Inhibits the Erythropoietic Growth and Kit Protein Expression (FIG. 3B)

c-kit and its ligand KL play an essential role in proliferation, differentiation, and survival of erythroid progenitor cells (24). Since c-kit expression is modulated by miR221 and miR222, we sought to determine whether proliferation and differentiation could be affected by the over-expression of miR221 and miR222 in a unilineage erythropoietic culture of purified CD34+ progenitor cells. The purified HPCs were grown in unilineage erythroid liquid suspension culture in the presence of KL and transfected on day 4 of differentiation with miR221, miR222 and the negative control dsRNAs at a concentration of 160 nM. Transfection efficiency, monitored with the FITC-conjugated miRs (see above), showed that 78% of cells were FITC-positive (data not shown); furthermore, apoptosis analysis showed that no toxicity was associated with miRNA transfection in these cells (data not shown).

Cells were grown in 24-well plates, counted, and diluted to a concentration of 2×10⁵ cell/ml every 2-3 days. As shown in FIG. 3B (left panel), cells transfected with miR221, miR222 or miR221 plus miR222 show a dramatic decrease in proliferation rate compared to the non transfected or control miR transfected cells. We also evaluated the effect of miR221 and miR222 on erythroid differentiation by analysing cell morphology at different stages of maturation. As shown in FIG. 3B (right panel), cells transfected with miR221, miR222, or miR221 plus miR222 differentiate more rapidly compared to non transfected or control miRNA transfected cells as demonstrated by the increased percentage of mature erythroblasts (polychromatophil and orthochromatic with respect to the total cells). Finally, we analysed c-kit expression in erythroid culture over-expressing miR221, miR222, miR221 plus miR222 or control miR two and four days after transfection (corresponding to day 6 and day 8 of erythroid differentiation, respectively). Western blotting analysis with a specific anti c-kit antibody showed a substantial decrease of c-kit protein expression in cells transfected with miR221, miR222 or miR221 plus miR222 (FIG. 3B, bottom panel). The protein expression levels of actin were unaffected by the transfected miR221, miR222, miR221 plus miR222 or control miRNA, indicating that miR221 and miR222 regulation is specific to c-kit. C-kit expression analysis nine or eleven days after transfection showed no down-modulation of protein levels maybe because of degradation and/or depletion of miRNAs (data not shown).

Lentiviral Gene Transfer of miR221 and miR222 in CD34+ Cells Erythropoietic Culture Acts as Growth Repressor and c-Kit Repressor (FIG. 3c)

Purified CD34+ cells were first incubated in erythroid cell culture medium containing or not KL and then transduced with a lentivirus containing either the empty vector (TWEEN) or the miR221 or miR222 through two viral infection cycles. Two days later the infection efficiency was controlled through flow cytometry analysis of GFP fluorescence and GFP positive cells were sorted. Sorted GFP+ cells were then grown in liquid suspension in erythroid cell culture medium at an initial cell density of 1×10⁵ cells/ml. Every two days the number of viable cells was determined and the morphology of the cells was controlled after cytocentrifugation and staining with May-Grünwald-Giemsa. When the cells reached a concentration of 8×10⁵ cells/ml or higher, were brought back to the initial cell concentration of 2×10⁵ cells/ml by adding fresh medium. As shown in FIG. 3C (left panel), HPCs transduced either with miR221 or miR222 exhibited a dramatic decrement of their growth rate, compared to those transduced with the empty vector (TWEEN). In parallel, the effect of miR221 or miR222 on erythroid differentiation/maturation was also evaluated. As reported in FIG. 3C (right panel), cells transduced with either miR221 or miR222 show an accelerated kinetics of erythroid cell maturation, compared to the cells transduced with the empty vector %. Furthermore, it is important to note that in miR221 and miR222-transduced cells an increased rate of cell death is observed at late days of culture (day 20-25), after reaching terminal maturation.

Moreover, down-modulation of c-kit expression was demonstrated by Western blot in erythroid precursors, transduced with miR222, at day 10 of unilineage culture.

miR221 and miR222 Impair CD34+ Engraftment Capacity in Xenotransplantation of Nod-SCID Mice (FIG. 4)

As shown in a representative experiment, CB CD34+ cells treated with miR221 or miR222 oligomers show a marked decrease of stem cell repopulating activity in NOD-SCID mice, as evaluated in terms of human CD45+ cell engraftment in the BM. Analysis of multilineage engraftment showed that all haematopoietic lineages, as well as B lymphocyte production, were down-modulated upon miR221 or miR222 oligomer transfection (results not shown). Furthermore, control studies confirmed that miR221 or miR222 oligomers transfection induced a significant down-modulation of c-kit protein in CD34+ cells maintained in erythroid culture (not shown), as observed in the other CD34+ cell transfection studies presented above (FIG. 3B).

Knockdown of miR 221 and 222 by Antisense Oligonucleotides Upmodulates Kit Protein Level by Unblocking Translation of Kit mRNA (FIG. 5)

To demonstrate the functional effect of the inhibition of the two microRNAs on the translation of kit mRNA, we transfected the TF-1 cell line (which expresses the c-kit protein) with the Anti-miRNA-221 or the Anti-miRNA-222, and compared the total c-kit protein expression level by Western Blot to a control consisting of TF-1 cells transfected with an Anti-miR Inhibitor-Negative control.

Specifically, TF-1 cells (5×10⁵ cells per well) supplemented with GM-CSF (5 ng/ml), were transfected with either Anti-miR miRNA Inhibitor negative control (Ambion, Austin, Tex.), Anti-miR-2211 Inhibitor (Ambion) or Anti-miR-222 Inhibitor (Ambion) at a final concentration of 250 nM, using Lipofectamine 2000 (Invitrogen) as transfection agent. At 72 h after transfection, immunoblotting using kit Ab (R&D) was performed by standard methods.

At 72 h post-transfection, the anti-miR oligonucleotides sharply enhanced the kit protein level (see Fig. below), whereas kit mRNA level was unmodified and miR 221 and 222 were sharply downmodulated (not shown). Our data demonstrate that anti-miR-221 and 222 treatments knock down miR 221 and 222 and upmodulates kit protein by unblocking kit mRNA translation. This is shown in FIG. 5, see the figure legends section below.

miR 130a and 130b Interact with the 3′UTR of Kit mRNA and Inhibit the Translation of the Messenger (FIG. 6 and Results not Shown)

To demonstrate the direct interaction between miR 130a and Kit mRNA, we inserted downstream the luciferase ORF the 1,800 bp 3′UTR of Kit mRNA. This reporter vector was cotransfected in the TF-1 cell line, with: (a) a control non-targeting RNA oligonucleotide, or (b) miR 130a and/or 222 oligonucleotides. Specifically, TF-1 cells (1×10⁵ cells/well), supplemented with GM-CSF (5 ng/ml), were co-transfected with 0.8 μg of pGL3-3′-UTR plasmid, 50 ng of Renilla and 20 pmol of either a stability-enhanced non-targeting RNA control oligonucleotide (Dharmacon, Lafayette, Colo.), or stability-enhanced miR 222 and/or 130a oligonucleotides (Dharmacon), all combined with Lipofectamine 2000 (Invitrogen). After 48 h cells were washed and lysed with the Passive Lysis Buffer (Promega), and their luciferase activity was measured using the Femtomaster FB 12 (Zylux, Oak Ridge, Tenn.). The relative reporter activity was obtained by normalization to the pGL3-3′-UTR/control oligonucleotide cotransfection.

The relative luciferase activity was markedly diminished following miR 130a and/or 222 co-transfection, as compared to the control RNA, indicating that the two miRs interfere with Kit mRNA translation via direct interaction with the 3′ UTR. These results are shown in FIG. 6. Refer to the figure legend section below.

The same effect was observed for miR 130b (results not shown), which includes the same seed sequence as miR 130a (FIG. 8).

### FIGURE LEGENDS

FIG. 1

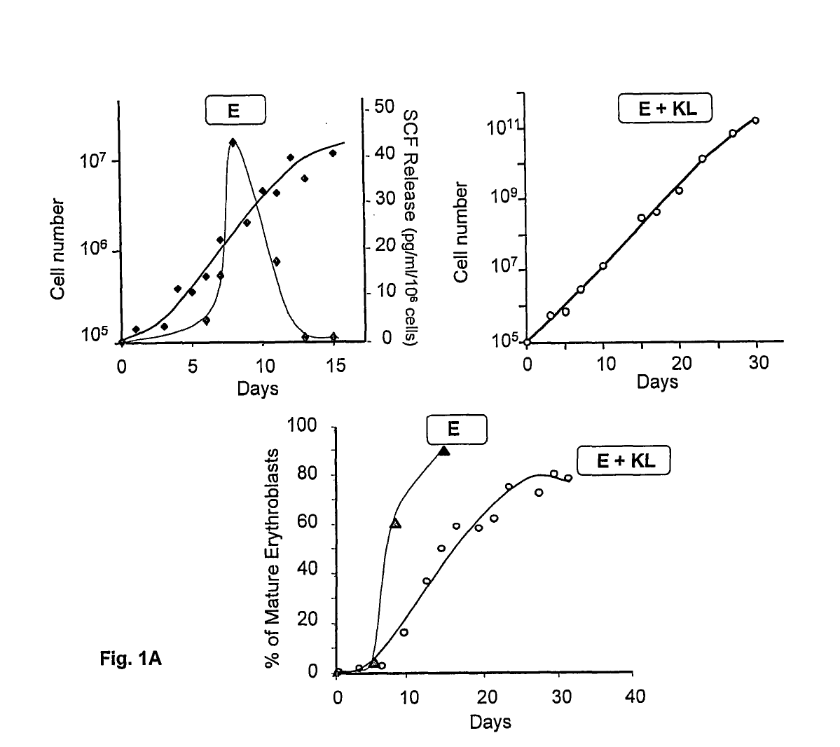

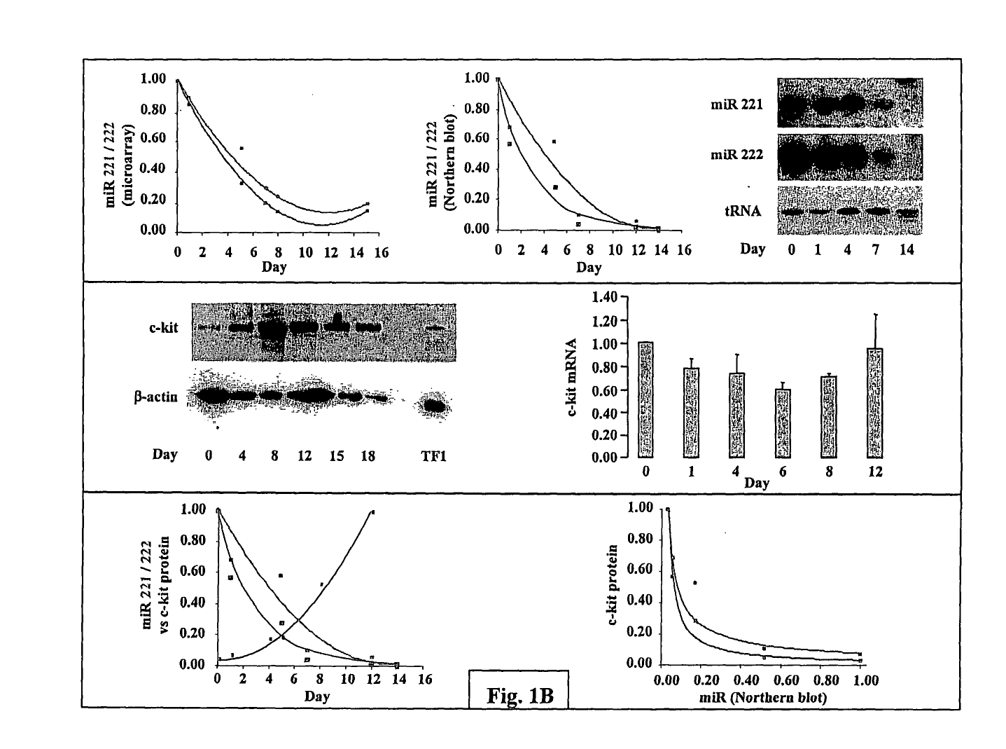

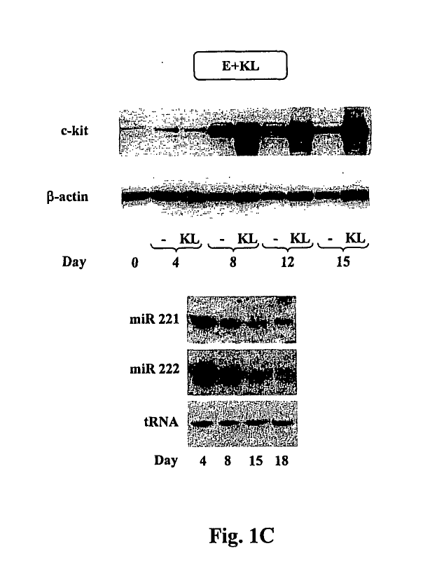

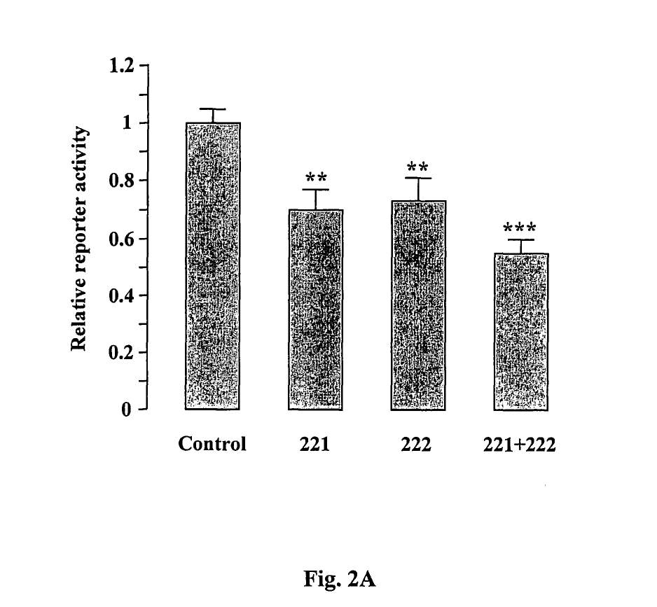

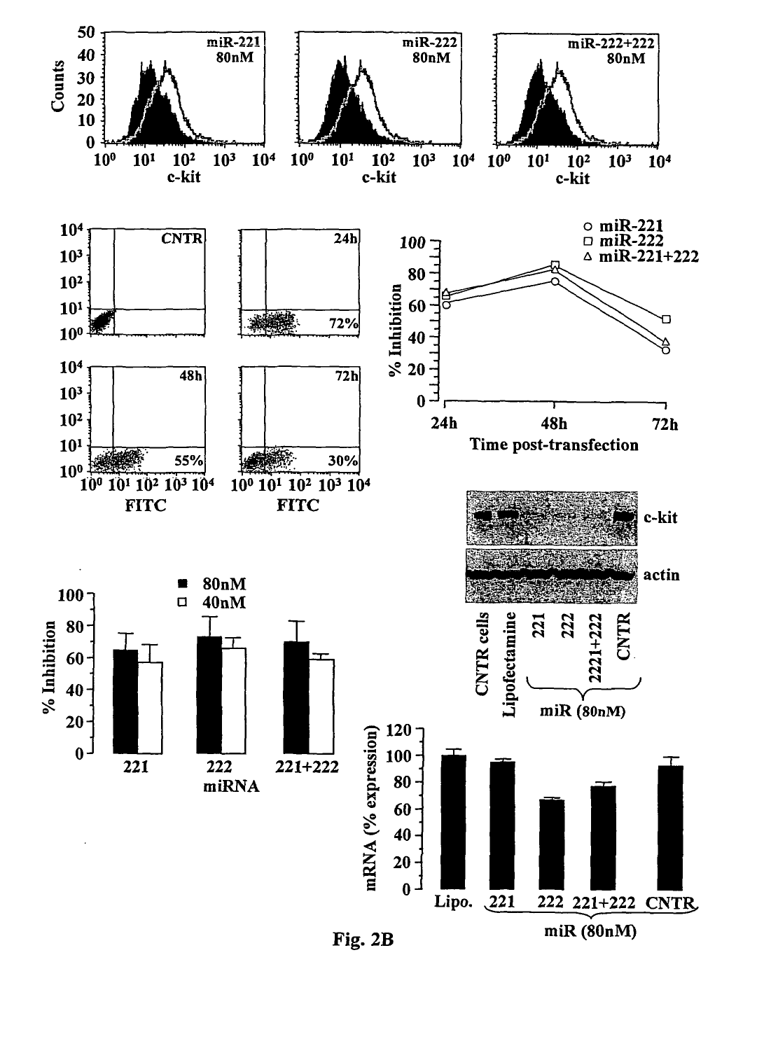

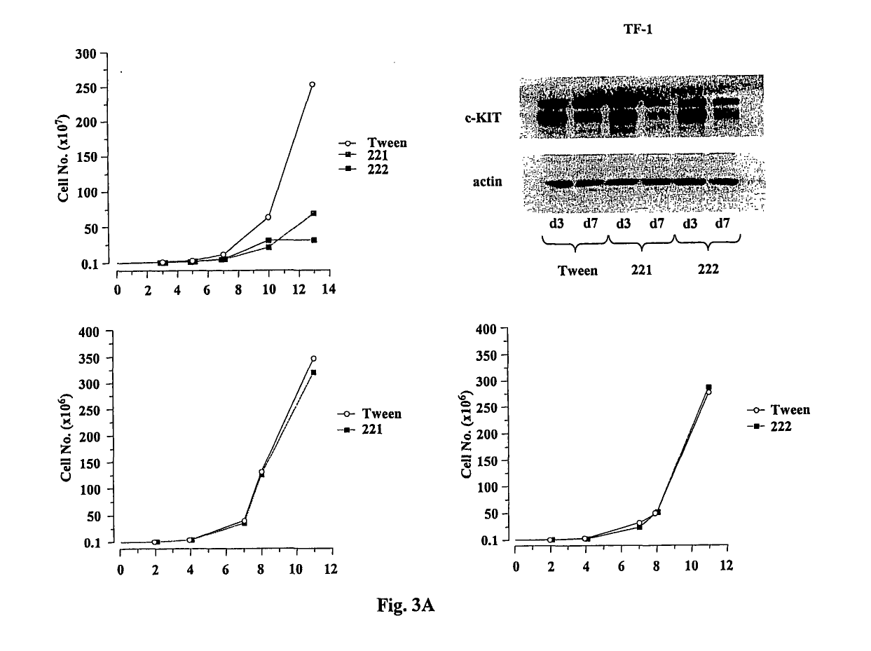

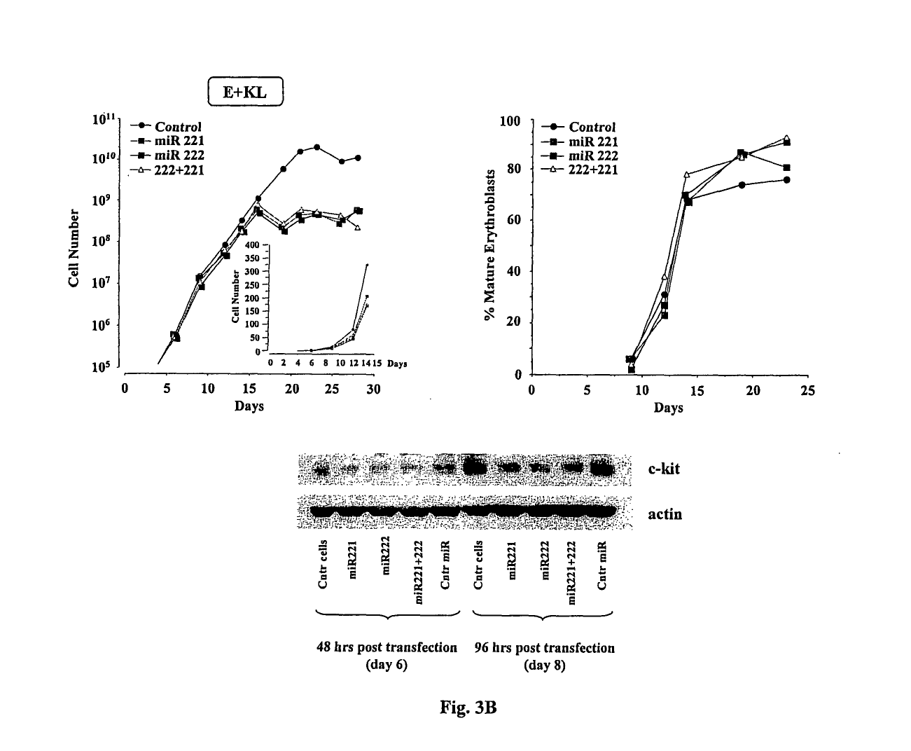

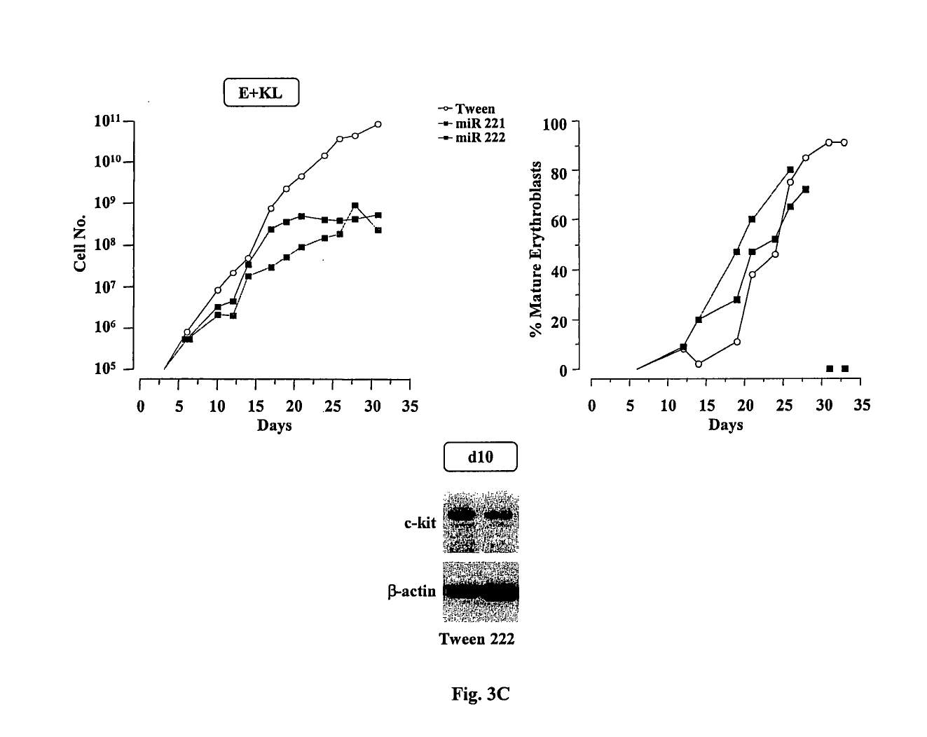

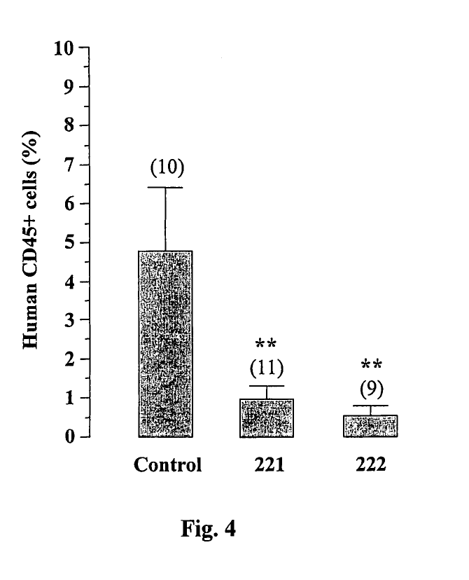

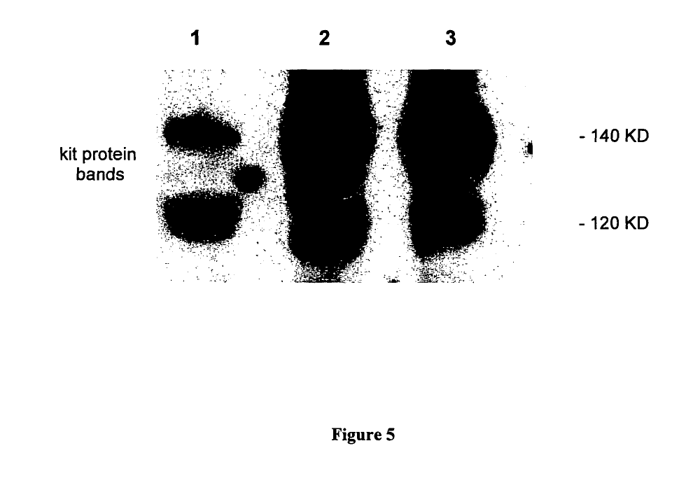

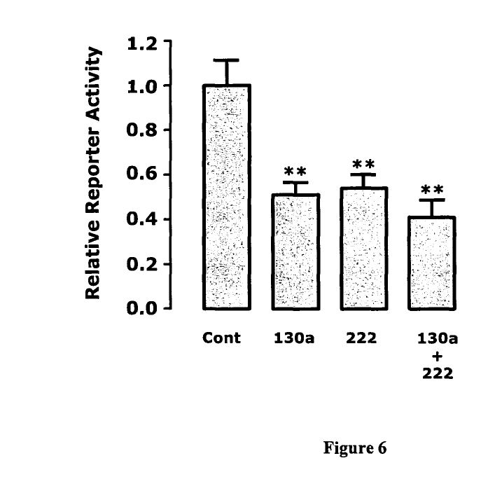

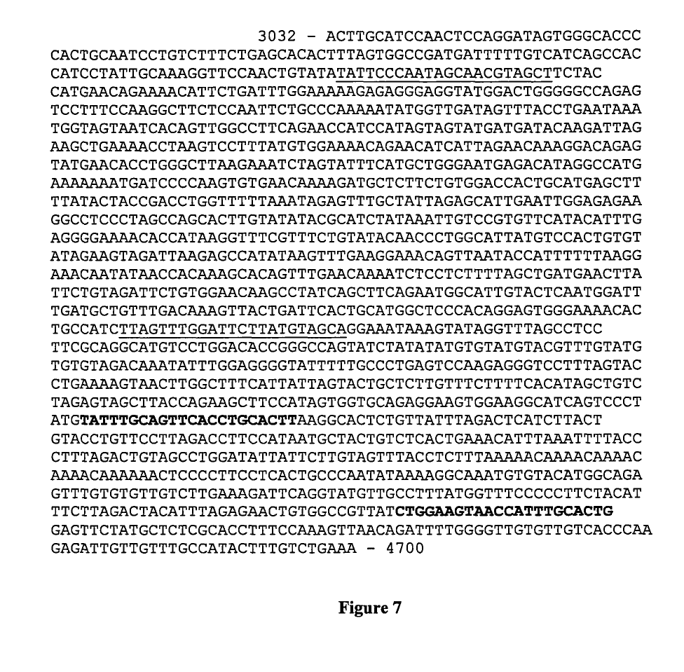

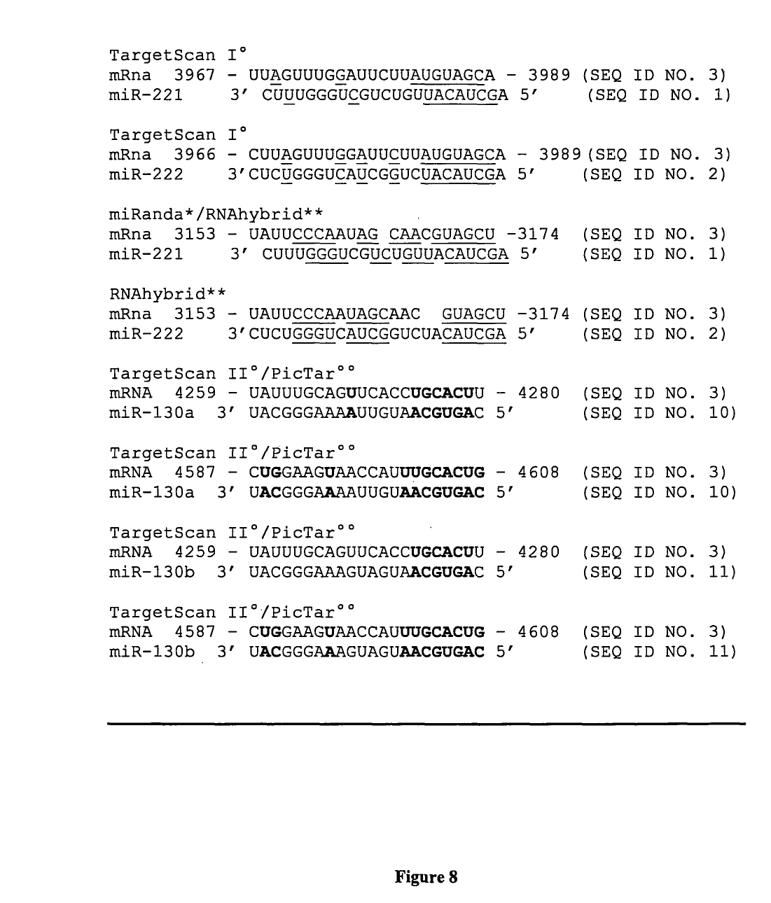

A Left panel. Growth curve and SCF release from bulk HPC erythroid cultures supplemented with Epo alone. 10⁵ purified CD34+ cells have been grown in liquid suspension cultures under selective erythroid conditions and at different days of culture total cell number and SCF concentration in culture supernatants was evaluated. The data represent mean values observed in 7 separate experiments.

Right Panel: Growth curve from bulk HPC erythroid cultures supplemented with Epo+KL. The data reported in the figure represent mean values of total cell number observed in 3 separate experiments.

Bottom Panel: kinetics of erythroid maturation of bulk HPC erythroid cultures supplemented with Epo alone (E) or Epo+KL (E+KL).

The data shown in the figure represent the percentage of mature erythroblasts (polychromatophilic+orthochromatic) at different days of culture.

B Top panel. miR221 and miR222 expression during erythroid differentiation: Microarray analysis and Northern blot revealed a remarkable down-regulation of miR expression during CD34+ erythroid maturation. The expression values were normalised as described in Materials and Methods and reported as ratios with respect to day 0.

Middle panel. C-kit expression during erythroid differentiation: the protein level, as seen by immunoblotting (left), reaches the highest level at late stages of erythropoiesis (day 12). β-actin protein was used to normalise the amount of loaded protein. Real time PCR analysis shows that c-kit mRNA level remains relatively constant during the entire maturation process (right).

Bottom panel. Inverse correlation representing miR expression versus c-kit protein level (miR221/c-kit: r² 0.93; miR222/c-kit r² 0.97).

C Top panel. Cord blood purified CD34+ cells were cultivated in standard erythroid medium±100 ng/ml SCF. Western blot revealed that c-kit protein was up-regulated in erythroblasts treated with SCF. β-actin protein was used to normalise the amount of loaded protein.

Bottom panel. miR221 and miR222, as seen by Northern blot, gradually decrease during erythroid differentiation in the presence of SCF.

FIG. 2

A miR221 and miR222 physically interact with c-kit 3′UTR. Reporter activity was normalised to the cotransfection between the empty Tween and the pGL3-3′UTR construct (first column). miR221 (second column) and miR222 (third column) cotransfection, together with the triple transfection with both the miRs (forth column), showed a remarkable decrease of luciferase-3′UTR mRNA translation, indicating an effective, pairing-dependent, repression by the miRs. Data is presented as mean+/−SD.

B c-kit expression analysis in TF1 cells transfected with miR221 or miR222 oligomers.

a. FACS analysis of membrane-bound c-kit expression in TF1 cells transfected with miR221, miR222, miR221 plus miR222 (filled histograms) compared to cells transfected with control miRNA (empty histogram) at a concentration of 80 nM 48 hours after transfection. C-kit expression levels in non-transfected or Lipofectamine treated cells were comparable to the ones observed in control miRNA transfected cells (data not shown).

b. FACS analysis of TF1 cells transfected with unconjugated (cont) or FITC-conjugated miR221 at a concentration of 80 nM or 40 nM. The percentage of FITC-positive cells is reported.

c. Percentage of c-kit expression inhibition in cells transfected with miR221, miR222, miR221 plus miR222 compared to cells transfected with control miR at a concentration of 80 nM (black bar) or 40 nM (white bar). Each point represents the mean and the standard error from four independent experiments.

d. Time response of c-kit expression inhibition in cells transfected with miR221, miR222, miR221 plus miR222 compared to control miRNAs 24, 48, and 72 hours after transfection. miRNAs were transfected at a concentration of 80 nM.

e. Western blotting analysis of c-kit expression in non-transfected (cont cells), Lipofectamine treated (Lipof.) or miRNA transfected TF 1 cells. Oligos were transfected at a concentration of 80 nM. Cell extract from controls or transfected cells was subjected to SDS-PAGE and Western blotting with an anti-kit antibody followed by an anti-goat HRP conjugated antibody. After developing with ECL, the filter was stripped and incubated with an anti-actin antibody followed by and anti-mouse HRP conjugated secondary antibody.

f. Real-time PCR was performed on cDNA amplified from RNA extracted from Lipofectamine-treated or miRNA transfected TF1 cells 48 hours after transfection. Values are reported as a percentage of c-kit mRNA expression in transfected cells compared to Lipofectamine treated cells (Lipof.) set as 100%. C-kit mRNA expression was normalised to the GAPDH mRNA expression. Each point represents the mean and the standard error from three measurements.

FIG. 3

A miR221 miR222 over-expression impairs cell growth of TF-1 erythroleukemic cells, expressing the c-kit receptor.

Upper left panel. Growth curve of TF-1 cells infected alternatively with the empty Tween, Tween-miR221 and Tween-miR222. (Upper Right panel) Western blotting using an antibody against the c-kit protein. Immunoblotting was performed on infected cells at day 3 and 7. At day 7 c-kit is down-modulated in the miRs infected cells

Lower panels. As a control of the specificity of cell growth inhibition, HL-60 cells, which do not express c-kit, were used. Accordingly the growth rate of the HL-60 cell line infected with the miRs, compared to the Tween alone, remains unaffected.

B Transfection of CD34+ progenitor cells cultured in erythroid medium plus KL with miRNAs

Proliferation curve (left panel) differentiation analysis (right panel) and c-kit expression (bottom panel) in HPC grown in unilineage erythropoietic culture (plus SCF, 100 ng/ml) and transfected with miR221, miR222, miR221 plus miR222, or control miRNA at a concentration of 160 nM on day 4 of erythroid differentiation (indicated by the arrow). Cells were counted every 2-3 days; cell number is reported in logarithmic scale (left panel). Percentage of mature erythroblasts (polychromatophil and orthochromatic with respect to total cells) is reported (right panel). C-kit expression (bottom panel) was analysed by Western blotting 48 and 96 hours after transfection (corresponding to day 6 and day 8 of erythroid differentiation, respectively) with an anti-kit specific antibody followed by an anti-goat HRP conjugated antibody. After developing with ECL, the filter was stripped and incubated with an anti-actin antibody followed by and anti-mouse HRP conjugated secondary antibody.

C Growth curve and kinetics of maturation of HPCs first transduced either with empty lentivirus (Tween) or with lentivirus containing miR221 or miR222 and then grown in erythroid cell cultures supplemented with KL.

In the left panel the total cell number at different days of culture of HPCs transduced either with the empty vector (Tween) or with miR221 or miR222 in reported (mean value in three separate experiments).

In the right panel the kinetics of erythroid maturation, with the percentage of the different types of erythroblast (proerythroblast, basophilic, polychromatophilic and orthochromatic erythroblasts) is reported (mean value in three separate experiments).

Western blot shows that c-kit is down-modulated in the miR222 infected erythroblasts at day 10 of unilineage differentiation. β-actin protein was used to normalise the amount of loaded protein.

FIG. 4

Engraftment of CD34+ cells transfected with miR221 and miR222 oligomer, following transplantation in NOD-SCID mice, as evaluated on the basis of the frequency of human CD45+ cells in recipient BM.

FIG. 5

Western Blot showing kit protein bands, on a total load of 20 μg of protein extract, 72 h post-transfection. Lane 1, kit protein in cells transfected with anti-miR inhibitor negative control. Lane 2 and lane 3, kit protein in anti-miR-221 and anti-miR-222 transfected cells respectively.

FIG. 6

miR 222 and 130a physically interact with Kit 3′UTR, as evaluated by luciferase targeting assay. Mean±SEM values from 9 separate experiments; **P<0.01 when compared to control. This shows that treatment with anti-miR 221 and 222 sequences markedly upmodulates kit protein. The same action has been shown by infecting the cells with “decoy” sequences in lentiviral vector (these sequences include the “seed” sequence matching 221/222, as well as the closeby “ancillary” matches). Noteworthily, these antisense “decoy” sequences match for <50% miR 221/222. The effect of anti-miR 221 and 222 sequences on kit protein level has been validated in functional assays (similar or identical to those presented in the Examples for miR 221 and 222).

The same results have been obtained with anti-miR 130a or 130b sequences (not shown).

FIG. 7

FIG. 7 shows the sequence of kit mRNA 3′UTR (as per Seq ID No. 3), including the sequence complementary to miR-221/222 (underlined) and miR-130a and -130b (highlighted in bold).

FIG. 8

Bioinformatic analysis according to different algorithms (as specified) suggests that miR 221 and 222, as well as miR 130a and -130b, have diverse target sequences in this 3′ UTR. This bioinformatic analysis also indicated that the target sequences in 3′ UTR comprise “seed” sequences (in red or bold) matching exactly the corresponding miR, coupled with ancillary nearby matches (also in red or bold). This bioinformatic analysis is in line with the luciferase assay results indicating that: (a) 221 and 222 directly interact with the 3′ UTR (original patent, FIG. 2A); (b) miR 130a does the same (see FIG. 5). (c) miR 130b (almost identical to miR 130b except for 2 nucleotides) does the same too (results not shown).

### Key to FIG. 8:

* http://cbio.mskcc.org/mirnaviewer
** http://www.rnaiweb.com
∘ http://genes.mit.edu/targetscan
∘∘ http://pictar.bio.nyu.edu

### REFERENCES

1. Bartel, D. P. (2004) Cell 116, 281-297
2. Lee, Y., Ahn, C., Han, J., Choi, H., Kim, J., Yim, J., Lee, J., Provost, P., Radmark, O., Kim, S. and Kim, V. N. (2003)Nature 425, 415-419
3. Yi, R., Qin, Y., Macara, I. G. and Cullen, B. R. (2003) Genes Dev. 17, 30011-3016
4. Ambros, V. (2004) Nature 431, 350-355
5. Yekta, S., Shih, I. H., Bartel, D. P. (2004) Science 304, 594-596
6. Lagos-Quintana, M., Rauhut, R., Lendeckel, W., Tuschl, T. (2001) Science 294, 853-858
7. Lagos-Quintana, M., Rauhut, R., Meyer, J., Borkhardt, A., Tuschl, T. (2003) RNA 9, 175-179
8. Houbaviy, H. B., Murray, M. F., Sharp, P. A. (2003) Dev Cell 5, 351-358
9. Lim, L. P., Glasner, M. E., Yekta, S., Burge, C. B., Bartel, D. P. (2003) Science 299, 1540
10. Sempere, L. F., Freemantle, S., Pitha-Rowe, I., Moss, E., Dmitrowsky, E., Ambros, V. (2004) Genome Biol 5, R13
11. Xu, P., Guo, M., Hay, B. A. (2004) Trends Genet. 20, 617-24
12. Cheng, A. M., Byrom, M. W., Shelton, J., Ford, L. P. (2005) Nucleic Acids Res 33, 1290-1297
13. Chen, C. Z., Li, L., Lodish, H. F., Bartel, D. P. (2004) Science 303, 83-86
14. Calin, G. A., Sevignani, C., Dumitru, C. D., Hyslop, T., Noch, E., Yendamuri, S., Shimizu, M., Rattan, S., Bullrich, F., Negrini, M., Croce, C. M. (2003) Proc Natl Acad Sci USA 101, 2999-3004
15. Dresios, J., Aschrafi, A., Owens, G. C., Vanderklish, P. W., Edelman G. M., Mauro, V. P. (2005) Proc Natl Acad Sci USA 102, 1865-1870.
16. Johnson, S. M., Grosshans, H., Shingara, J., Byrom, M., Jarvis, R., Cheng, A., Labourier, E., Reinert, K. L., Brown, D., Slack, F. J. (2005) Cell 120, 635-647
17. Liu, C. G., Calin, G. A., Meloon, B., Gamliel, N., Sevignani, C., Ferracin, M., Dumitru, C. D., Shimizu, M., Zupo, S., Dono, M., Adler, H., Bullrich, F., Negrini, M., Croce, C. M. (2004) Proc Natl Acad Sci USA 101, 9740-4
18. Chomczynski, P., Sacchi, N. (1987) Anal Biochem 162, 156-159
19. Livak, K. J., Schmittgen, T. D. (2001) Methods 25, 402-408
20. Bonci, D., Cittadini, A., Latronico, M. V. G., Borello, U., Aycock, J. K., Drusco, A., Innocenzi, A., Follenzi, A., Lavitrano, M., Monti, M. G., RossJr, J., Naldini, L., Peschle, C., Cossu, G., Condorelli, G. (2003) Gene Ther 10, 630-636
21. Ricci-Vitiani, L., Pedini, F., Collinari, C., Condorelli, G., Bonci, D., Bez, A., Colombo, A., Parati, E., Peschle, C., De Maria, R. (2004) J Exp Med 200, 1275-1266
22. Ziegler, B., Valtieri, M., Almeida Porada, G., De Maria, R., Müller, R., Masella, B., Gabbianelli, M., Casella, I., Pelosi, E., Bock, T., Zanjani, E. D., Peschle C. (1999) Science 285, 1553-1558
23. Vitelli, L., Condorelli, G. L., Lulli, V., Hoang, T., Luchetti, L., Croce, C. M., Peschle, C. (2000)Mol Cell Biol 20, 5330-5342
24. Huang, E., Nocka, K., Beier, D. R., Chu, T. Y., Buck, J., Lahm, H. W., Wellner, D., Leder, P., Besmer, P. (1990) Cell 63, 225-233

The disclosure of all citations and publication is hereby incorporated by reference.

### Explanation of Sequence Listing (all Sequences are 5′-3′ Unless Otherwise Apparent)

SEQ. ID NO. 1

miR 221 (wt mature microRNA)
agcuacauugucugcuggguuuc

SEQ. ID NO. 2

miR 222 (wt mature microRNA)
agcuacaucuggcuacugggucuc

SEQ. ID NO. 3

c-kit 3′UTR (inserted in pGL3 vector used for the
luciferase assay; inserted in lentiviral
vector and used as an anti-miR 221-222 “decoy”)

SEQ. ID NO. 4

(top strand, complementary strand is SEQ ID
NO. 23)
miR-221 oligomer (used for cell transfection)
5′ucgauguaacagacgacccaaag3′

(SEQ ID NO. 4)

3′agcuacauugucugcuggguuuc-5′
(5′CUUUGGGUCGUCUGUUACAUCGA 3′ SEQ ID NO. 23)

SEQ. ID NO. 5

(top strand, complementary strand is SEQ ID
NO. 24)
miR-222 oligomer (used for cell transfection)
5′-ucgauguagaccgaugacccagag-3′
3′-agcuacaucuggcuacugggucuc-5′
(5′CUCUGGGUCAUCGGUCUACAUCGA3′, SEQ ID NO. 24)

SEQ. ID NO. 6

miR-221 precursor (inserted in Lentivirus vector
for cell infection)

SEQ. ID NO. 7

miR-222 precursor (inserted in Lentivirus vector
for cell infection)

SEQ. ID NO. 8

Anti221 2′-O-methyloligonucleotide (used for cell
transfection as anti-miR)
Uaaauuuuacccuuuagacuguagccuggau

SEQ. ID NO. 9

Anti222 2′-O-methyloligonucleotide (used for cell
transfection as anti-miR)
Acagagacuuggcagccagaaauauccuccu

SEQ. ID NO. 10

miR-130a oligomer (wt mature microRNA)
cagugcaauguuaaaagggcau

SEQ.ID NO. 11

miR-130b oligomer (wt mature microRNA)
cagugcaaugaugaaagggcau

SEQ. ID NO. 12

(top strand, complementary strand is
SEQ ID NO. 25)
miR-130a oligomer (used for cell transfection)
5′-gucacguuacaauuuucccgua-3′
3′-cagugcaauguuaaaagggcau-5′
(5′-UACGGGAAAAUUGUAACGUGAC-3′, SEQ ID NO. 25)

SEQ. ID NO. 13

miR-130b oligomer (used for cell transfection)
(top strand, complementary strand is SEQ ID NO. 26)
5′-gucacguuacuacuuucccgua-3′
3′-cagugcaaugaugaaagggcau-5′
(5′-UACGGGAAAGUAGUAACGUGAC-3′ SEQ ID NO. 26)
The probes and primers are discussed in the
Examples.
Anti-miR-130a

SEQ. ID NO. 27

5′-AUGCCCUUUUAACAUUGCACUG-3′,
anti-miR-130b

SEQ. ID NO. 28

5′-AUGCCCUUUCAUCAUUGCACUG-3′

1. A therapeutic method comprising administering to a patient in need thereof an effective amount of antisense RNA specific for all or part of the 3′ untranslated region of kit protein mRNA, wherein the antisense RNA is a micro RNA.

2. The method of claim 1, wherein the therapy is treatment of cancer.

3. The method of claim 1, wherein the therapy is treatment of GIST (gastro-intestinal stromal tumour), kit-dependent acute leukaemias, erythroleukemia, papillary thyroid carcinoma, or other kit-dependent tumours or disease conditions.

4. The method of claim 1, wherein the therapy is the modulation of erythropoiesis.

5. The method of claim 1, wherein said antisense RNA is effective by kit receptor down-modulation.

6. The method of claim 1, wherein the antisense RNA is specific for all or part of the 3′ untranslated region of kit protein mRNA.

7. The method of claim 1, wherein the antisense RNA has at least 60% homology with a selected region of the 3′ untranslated region of kit protein mRNA.

8. The method of claim 1, wherein the antisense RNA is between about 12 bases and 45 bases in length.

9. The method of claim 1, wherein the antisense RNA is selected from the group consisting of miR 221 (SEQ ID NO:1), miR 222 (SEQ ID NO:2) miR 130a (SEQ ID NO:10), and 130b (SEQ ID NO:11).

10. The method of claim 9, wherein the antisense RNA is miR 221 (SEQ ID NO:1) or miR 222 (SEQ ID NO:2).

11. The method of claim 1, wherein the antisense RNA is a mutant or variant of miR221, miR222, miR130a or miR130b.

12. An inhibitor or suppressor of miR 221, miR 222, miR130a or miR130b.

13. An inhibitor or suppressor according to claim 12, which is a sense RNA.

14. A therapeutic method comprising administering to a patient in need thereof an effective amount of an inhibitor or suppressor according to claim 12.

15. The method of claim 14, wherein the therapy comprises use of at least two of:
an inhibitor for miR221,
an inhibitor for miR222,
an inhibitor for miR130a or
an inhibitor for miR130b.

16. The method of claim 14, wherein the therapy is for suppressed haematopoiesis in cancer patients and β-thalassemia and other β-haemoglobin diseases.

17. The method of claim 14, wherein the therapy is for the potentiation of ex vivo expansion of haematopoietic stem/progenitor cells or for the enhancement of the proliferative and anti-apoptotic effects of kit in non-haematopoietic cells.

18. The method of claim 14, wherein the inhibitor or suppressor is selected from the group consisting of: SEQ ID NO:8, SEQ ID NO:9, SEQ ID NO:27 and SEQ ID NO:28.

19. A vector comprising RNA or DNA encoding antisense RNA specific for all or part of the 3′ untranslated region of kit protein mRNA, wherein the antisense RNA is a micro RNA.

20. The vector of claim 19, which encodes or comprises the mature form of the RNA, where the RNA is a micro RNA.

21. A method of treating GIST (gastro-intestinal stromal tumour), kit-dependent acute leukaemias, erythroleukemia, or other kit-dependent tumours or disease conditions, comprising administering to a patient antisense RNA specific for all or part of the 3′ untranslated region of kit protein mRNA, wherein the antisense RNA is a micro RNA.

22. A method of treating suppressed haematopoiesis in cancer patients and β-thalassemia and other β-haemoglobin diseases, or for the potentiation of ex vivo expansion of haematopoietic stem/progenitor cells or for the enhancement of the proliferative and anti-apoptotic effects of kit in non-haematopoietic cells, comprising administering to a patient the inhibitor or suppressor of claim 12.
